SEQUENCE LISTING (4 sequences)

SEQ ID NO: 1 - DNA, 1245 bp, Sphingomonas elodea
atggaaccggagaccatcctcatgtcggacaccaccgcagtcgaccgatctccggtaaagtcaggcctacgtttttcggccctagacagcctgcgcggcatctgcgcatgcatgatcgttctgttccaccttcgctccactggcgtcgtcacgaactcgcatctggtccgaaacagctggatgttcgtcgacttctttttcgtcctcagcggcttcgtcatcgcgtgcggctatctggagcgattgcgggagggctattccgtgcggcagttcatgctgctgcgcctgggccgggtctatccgttgcacctggccgtcctcctcctgttcgtggtgatcgagctagcgggggccatgctcggtaccgccgggctcagcgcccgcgccgccttttcggagccgcgaacccctgcggagctcgccggcacgctcgcgctggtccagatcttctgcggcttcccctcgatcgtctggaacggcccgagctggagcatcgctgcggaggtctggacctatctgctggtcgcgctcgtcgtgcgcgcgctgcccgggcgaaccgcatgggctgccacgggtctggcgcttgccgccttcgccacgctcgcgctcgccggtgcggccgcctgggacccggcgacgggctttgcctttgtccgctgcgtcctgggcttctcggtgggcgtgctgtgctggatcctgttctcggcaatggggcggccgaggatgggaaccgcgatcgcaacgatcttggagctggtggcggtcgcatcgtgctgcgcgctggtggcttcgggaagcctgccgctggcggcgccgatcgtgttcgccggcacggtgctgttgttcgcggccgagcagggcatggtcagtcggctcctgaagctcgcgcccttcctcgcgctcggcaccctctcctactcgatctacatggtgcacacgctggtgatcgcacgcagtctggacgtgctctcactcgcgggcaggctgttgcatcacccgctggtggagacacggctcggcagcggtggtacgatcaaggtgctggtgttcgcgccggacgcaatggcgttcgcggtgctcggcggcatcgtgctgtgttcggcgctcacctatcgctggatcgaggcgcccgcgcgggacctgtcgcgcgcactggtccgccagagcgggcggcgcggcagcttggcggccgccccggacgcagcacgcgaccccgaggccctgccggcaaccgccacgagctga

SEQ ID NO: 2 - protein, 414 aa, Sphingomonas elodea
MEPETILMSDTTAVDRSPVKSGLRFSALDSLRGICACMIVLFHLRSTGVVTNSHLVRNSWMFVDFFFVLSGFVIACGYLERLREGYSVRQFMLLRLGRVYPLHLAVLLLFVVIELAGAMLGTAGLSARAAFSEPRTPAELAGTLALVQIFCGFPSIVWNGPSWSIAAEVWTYLLVALVVRALPGRTAWAATGLALAAFATLALAGAAAWDPATGFAFVRCVLGFSVGVLCWILFSAMGRPRMGTAIATILELVAVASCCALVASGSLPLAAPIVFAGTVLLFAAEQGMVSRLLKLAPFLALGTLSYSIYMVHTLVIARSLDVLSLAGRLLHHPLVETRLGSGGTIKVLVFAPDAMAFAVLGGIVLCSALTYRWIEAPARDLSRALVRQSGRRGSLAAAPDAARDPEALPATATS

SEQ ID NO: 3 - DNA, 25 bp, Artificial Sequence
  primer_bind (1)..(25): PCR primer
ttggagctctctggacctatctgct

SEQ ID NO: 4 - DNA, 25 bp, Artificial Sequence
  primer_bind (1)..(25): PCR primer
gtttctagacttcaggagccgactg

(19) United States
(12) Patent Application Publication
(10) Pub. No.: US 2003/0100078 A1
(43) Pub. Date: May 29, 2003

(54) Mutant strain of Sphingomonas elodea which produces non-acetylated gellan gum

(76) Inventors: Nancy E. Harding, San Diego, CA (US); Yamini N. Patel, San Diego, CA (US); John O. McQuown, San Diego, CA (US)

Correspondence Address: FITZPATRICK CELLA HARPER & SCINTO, 30 ROCKEFELLER PLAZA, NEW YORK, NY, 10112, US

(21) Appl. No.: 10/188,092

(22) Filed: Jul. 3, 2002

Related U.S. Application Data
(60) Provisional application No. 60/302,787, filed on Jul. 3, 2001.

(51) Int. Cl. C12P 19/04; C08B 37/00; C12N 1/21

(52) U.S. Cl. 435/101; 435/252.3; 536/123

(57) ABSTRACT

This invention provides a mutant Sphingomonas microorganism which produces a polysaccharide polymer comprising repeating tetramer units having a D-glucose:D-glucuronic acid:L-rhamnose ratio of about 2:1:1, wherein the D-glucose moieties are linked in a β-[1,4] configuration to the D-glucuronic acid moiety. One of the D-glucose moieties is linked to the L-rhamnose moiety in an α-[1,3] configuration, the other glucose moiety is linked to the L-rhamnose moiety in a β-[1,4] configuration, wherein the polysaccharide polymer is substantially non-acetylated. This invention also provides the substantially non-acetylated polymer, as well as process for obtaining the substantially non-acetylated polymer.

This application claims the benefit of U.S. Provisional Application No. 60/302,787, filed on Jul. 3, 2001.

### BACKGROUND OF THE INVENTION

1. Field of the Invention

The present invention relates to a mutant strain of Sphingomonas elodea which produces non-acetylated gellan gum. The invention also relates to non-acetylated gellan gum.

2. Discussion of the Related Art

Polysaccharides or gums are primarily used to thicken or gel water and are frequently classified into two groups: thickeners and gelling agents. Typical thickeners include starches, xanthan gum, guar gum, carboxymethylcellulose, alginate, methylcellulose, gum karaya and gum tragacanth. Common gelling agents include gelatin, gellan gum, starch, alginate, pectin, carrageenan, agar and methylcellulose.

Gelling agents are used by the food industry in a variety of applications, including confectionery jellies, jams and jellies, dessert gels, icings and dairy products. Additionally, gelling agents may be used as components of microbiological media. Gelling agents differ in the conditions under which they can be used as well as in the texture of the gels they form. These distinctive properties of gels have led to the exclusive use of certain gelling agents in a number of products (e.g., starch in confectionery jellies; gelatin in capsules; agar in icings; and alginate in pimento strips).

Gellan gum (S-60) is produced by the microorganism Sphingomonas elodea (ATCC 31461) as disclosed in U.S. Pat. Nos. 4,377,636, 4,326,053, 4,326,052 and 4,385,123, the contents of which herein are incorporated by reference. Commercially, the gum is formed by inoculating a carefully formulated fermentation medium with a Sphingomonas organism. The fermentation medium contains a carbon source, phosphate, organic and inorganic nitrogen sources, and appropriate trace elements. The fermentation is carried out under sterile conditions with strict control of aeration, agitation, temperature and pH. When the fermentation is complete, the viscous broth is pasteurized to kill viable cells prior to recovery of the gum.

The gum can be recovered in several ways. Direct recovery from the broth yields the gum in its native or high acyl form. Recovery after deacylation by treatment with alkali provides the gum in its low acyl form. The acyl groups have a profound influence on gel characteristics. The gel characteristics of gellan gum have been altered by reducing the level of acyl group substitutions by chemical deacylation. The glyceryl group is believed to have a greater effect on gel propoerties than the acetyl group. Baird et al. (1992) Gellan Gum: Effect of Composition On Gel Properties, Phillips et al. (Eds.), Gums and Stabilizers for the Food Industry 6 (pp. 479-487), Oxford, Permagon Press.

The constituent sugars of gellan gum are D-glucose, D-glucuronic acid and L-rhamnose in the molar ratio of 2:1:1, which are linked together to give a primary structure consisting of a linear tetrasaccharide repeat unit in the following order: D-glucose:D-glucuronic acid:D-glucose:L-rhamnose. Jannson et al. (1983) Carbohydr. Res. 124:135-139; O'Neill et al. (1983) Carbohydr. Res. 124:123-133. In the native or high acyl form of gellan gum, two acyl substituents, acetate and glycerate, are present. Both substituents are located on the same glucose residue, and on average, there is one glycerate per repeat unit and one acetate for every two repeat units as shown below. Kuo et al. (1986) Carbohydr. Res. 156:173-187.

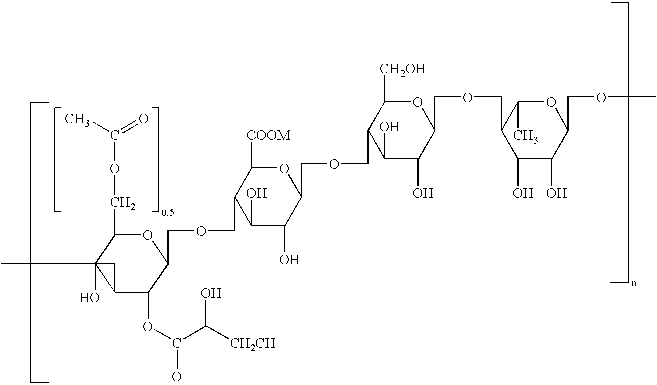

Sphingans are polysaccharides produced by bacteria of the genus Sphingomonas. U.S. Pat. No. 5,854,034 discloses a method for increasing production of sphingans in various strains of Sphingomonas. The disclosed method involves isolating sequences of DNA as segments from sphingan-producing bacteria, cloning the isolated segments, and incorporating multiple copies of the cloned segments into sphingan-producing or non-producing mutants of Sphingomonas bacteria. The patent does not disclose modification of acyl group substitution of gellan gum.

Chemical mutagenesis of gellan gum producing Sphingomonas paucimobilis bacteria has been reported. Jay et al. (1988) Carbohydr. Polymers 35:179-188. Several mutant strains of Sphingomonas paucimobilis, which produce non-acetylated, non-glycerylated and non-acylated gellan gum, respectively, reportedly were obtained. The article does not describe isolation or identification of the genes responsible for acetylation and glyceration of gellan gum in Sphingomonas bacteria. Moreover, experiments conducted by the inventors suggest that the mutant Sphingomonas paucimobilis obtained in Jay, et al. do not produce fully non-acetylated or non-glycerylated gellan gum.

To date, the predominant method utilized for gellan gum deacylation has been by hydrolysis under alkaline conditions. However, it has been found that chemical processes for deacylating gellan gum may result in a number of undesirable side effects that may cause hydrolysis of the polymer backbone, resulting in an irreversible change in the conformation of the molecule and lower molecular weight.

Modification of gellan gum has been described previously. For example, Baird et al., described methods for preparing chemically deacetylated gellan gum as well as chemically deacylated gellan gum. Baird et al. (1992) Gellan Gum: Effect of Composition On Gel Properties, Phillips et al. (Eds.) Gums and Stabilizers for the Food Industry 6 (pp. 479-487), Oxford, Permagon Press. Chemical deacylation of gellan gum produced by Sphingomonas elodea also is described in Kuo et al. (1986) Carbohydr. Res. 156:173-187.

It would be highly desirable to avoid chemical deacylation of gellan gum by obtaining and using mutant strains of Sphingomonas elodea microorganisms to produce non-acylated or non-acetylated gellan gum.

### SUMMARY OF THE INVENTION

The present invention provides a mutant Sphingomonas microorganism which produces a polysaccharide polymer comprising repeating tetramer units having a D-glucose:D-glucuronic acid:L-rhamnose ratio of about 2:1:1, wherein the D-glucose moieties are linked in a β-[1,4] configuration to the D-glucuronic acid moiety. One of the D-glucose moieties is linked to the L-rhamnose moiety in an α-[1,3] configuration, the other glucose moiety is linked to the L-rhamnose moiety in a β-[1,4] configuration, wherein the polysaccharide polymer is substantially non-acetylated.

The present invention also provides a polysaccharide polymer comprising repeating tetramer units having a D-glucose:D-glucuronic acid:L-rhamnose ratio of about 2:1:1, wherein the D-glucose moieties are linked in a β-[1,4] configuration to the D-glucuronic acid moiety. One of the glucose moieties is linked to the L-rhamnose moiety in an α-[1,3] configuration, the other glucose moiety is linked to the L-rhamnose moiety in a β-[1,4] configuration, wherein the polysaccharide polymer is substantially non-acetylated.

The present invention further provides a process for preparing a polysaccharide polymer comprising repeating tetramer units having a D-glucose:D-glucuronic acid, L-rhamnose ratio of about 2:1:1, wherein the D-glucose moieties are linked in a β-[1,4] configuration to the D-glucuronic acid moiety. One of the glucose moieties is linked to the L-rhamnose moiety in an α-[1,3] configuration, the other glucose moiety is linked to the L-rhamnose moiety in a β-[1,4] configuration, wherein the polysaccharide polymer is substantially non-acetylated. The process involves: (a) obtaining a mutant Sphingomonas which produces the substantially non-acetylated polysaccharide polymer; and (b) culturing the mutant Sphingomonas under conditions effective to produce the substantially non-acetylated polysaccharide polymer.

This invention is also directed to a DNA sequence which expresses the acetyl transferase gene of Sphingomonas and compositions comprising a Sphingomonas elodea polysaccharide polymer with substantially reduced levels of acetate.

### BRIEF DESCRIPTION OF THE DRAWINGS

FIG. 1 is a genetic and physical map of the DNA region of Sphingomonas elodea encoding acetyl transferase.

FIG. 2 shows the complete DNA sequence for the acetyl transferase gene.

### DETAILED DESCRIPTION OF THE INVENTION

As used herein, the term “non-acetylated” refers to a non-native form of a polysaccharide which differs from its native form in that it is produced with a substantially reduced level of acetyl substitution. Thus, non-acetylated gellan gum differs from native gellan gum in that native gellan gum has, on average, about one acetyl group substituent every 2 repeat units, whereas the non-acetylated gellan gum of this invention is produced with substantially no acetyl substitution and no effect on glyceryl substitution. By introducing a null mutation in the acetyl transferase gene, the non-acetylated gellan gum of this invention substantially lacks acetyl groups. As used herein, the term “substantially reduced levels of acetyl substitution” refers to a polysaccharide containing acetyl content reduced by about 85%, preferably reduced by about 90%, and most preferably reduced by about 95%.

The non-acetylated gellan gum of this invention also differs from de-acetylated gellan gum in that de-acetylated gellan gum is produced by removing the acetyl substituent from native gellan gum, whereas non-acetylated gellan gum is produced such that the acetyl substituent is never added because the acetyl transferase gene is inactivated.

As used herein, the term “acetyl transferase deficient” refers to a non-native form of a strain of bacteria which differs from its native form in that it lacks a functional acetyl transferase gene. Thus, an acetyl transferase deficient strain of Sphingomonas elodea differs from naturally occurring Sphingomonas elodea in that naturally occurring Sphingomonas elodea has a functional acetyl transferase gene, whereas an acetyl transferase deficient Sphingomonas elodea does not have a functional acetyl transferase gene. A naturally occurring strain of Sphingomonas may be rendered “acetyl transferase deficient” by disabling, inactivating, removing or partially removing the acetyl transferase gene.

Significantly, the DNA sequence of Sphingomonas elodea that encodes acetyl transferase production has been identified. Acetyl transferase is responsible for acetylation of gellan gum. Accordingly, acetylation of the gellan gum in Sphingomonas elodea may be eliminated through mutations of the acetyl transferase gene using methods well known to those skilled in the art, such as, for example, point mutations, transposon mutagenesis, deletions, insertions, and the like. These procedures may be used to obtain the mutant strains of Sphingomonas elodea of this invention, which produce non-acetylated gellan gum.

The mutant Sphingomonas elodea of this invention produce non-acetylated gellan gum. The mutant Sphingomonas elodea of this invention can be grown under conditions generally known in the art for growth of wild type Sphingomonas elodea. For example, the mutants of this invention can be grown on suitable assimilable carbon sources, such as glucose, sucrose, maltose, starch, complex carbohydrates, such as molasses or corn syrup, various organic acids and the like. Mixtures of carbon sources may also be employed. The concentration of carbon sources supplied is often between 10 and 60 grams per liter (g/l). An assimilable source of organic or inorganic nitrogen also is necessary for growth, and is generally between about 0.1 and 10.0 g/l. Examples of suitable nitrogen sources are ammonia, ammonium salts, nitrate, urea, yeast extract, peptone or other hydrolyzed proteinaceous materials or mixtures thereof. Minerals also are necessary for growth. Examples of suitable minerals include phosphorus, sulfur, potassium, sodium, iron and magnesium.

Optimal temperatures for growth of the mutant Sphingomonas elodea of this invention generally are between about 25° C. and about 37° C., preferably between about 30° C. and about 36° C. The mutant Sphingomonas elodea cells may be grown aerobically by supplying sufficient air or oxygen so that an adequate level of dissolved oxygen is maintained. The pH generally is maintained at about 6 to about 8 and, preferably at about 6.5 to about 7.5.

The non-acetylated polysaccharides of the present invention may be recovered from fermentation broths by any suitable means. Such methods are known to those skilled in the art. For example, precipitation with isopropanol, ethanol or other suitable alcohol readily yields the substantially non-acetylated polysaccharides of this invention. Alternatively, the polymers may be recovered from the fermentation broth by ultra-filtration.

The non-acetylated polysaccharides of the present invention may be used as gelling agents in a variety of fluid food products including confectionery jellies, jams and jellies, dessert gels, icings and dairy products, such as, for example, ice cream, frozen yogurt, cottage cheese, sour cream, non-dairy frozen toppings and bakery fillings.

The present invention also provides compositions comprising a substantially non-acetylated gellan gum, water, a gelling salt and a sequestrant. The concentration of gelling salt in the compositions will vary depending upon the particular gelling salt used. For example, sodium and potassium gelling salts generally are used at concentrations ranging from about 0.020M to about 0.200M, while calcium and magnesium gelling salts typically are used at concentrations ranging from about 0.002M to about 0.015M. The amount of sequestrant used in the compositions typically ranges from about 0.05 percent to about 0.25 percent by weight.

When fully hydrated, the non-acetylated gellan gums of the present invention will form gels with many different ions. Preferably, the gelling salt is a calcium salt, a sodium salt or a potassium salt. Most preferably, the gelling salt is CaCl₂. Sodium citrate is the preferred sequestrant.

Yet another embodiment of this invention is directed to the DNA sequence encoding acetyl transferase of Sphingomonas elodea. The DNA sequence of this invention may be isolated from Sphingomonas strains using methods that are well known to those skilled in the art. Typically, the bacteria are cultured to produce a fermentation broth. The bacterial cells are centrifuged and suspended for DNA extraction. The DNA extraction process generally involves removing the proteins from the fermentation broths and then precipitating the DNA using a solvent such as, for example, isopropanol. The precipitated DNA fragments may then be treated with a restriction enzyme, for example, EcoRI or PstI, to produce smaller DNA fragments, which then may be cloned into appropriate vectors according to conventional methods that are well known to those skilled in the art.

The examples which follow are intended to illustrate some of the preferred embodiments of the present invention, and no limitation is implied.

### EXAMPLE 1

### Preparation of a Low Acetyl Mutant of Sphingomonas elodea

LAM-1 is a Low Acetyl Mutant of Sphingomonas elodea produced by chemical mutagenesis which blocks acetylation of gellan gum. LAM-1 produces gellan gum which is deficient in acetyl group substitution.

LAM-1 was produced by chemical mutagenesis of Sphingomonas elodea strain S-60wtc (S-60wtc is a derivative of strain S. elodea, ATCC 31461, which was selected as a spontaneous isolate with increased ability to take up plasmid DNA) under the following conditions:

| | |
|---|---|
| Buffer | TRIS |
| pH | 8 |
| EMS | 15 μl/ml |
| Time | 30 min. |
| Temp | 30° C. |

Several colonies from fresh plates were resuspended in 5.0 ml sterile deionized water. The suspension was shaken vigorously, centrifuged, and then suspended in 10 ml of buffer. Ethyl Methane Sulfonate (15 μl/ml) was added and the suspension was incubated on a roller drum for 30 minutes at 30° C. After incubation, the mutated cultures were centrifuged, washed once in buffer and resuspended. Aliquots were dispensed to YM flasks for expression. LAM-1 was isolated from the mutated cultures using screening procedures well known by those skilled in the art. The amount of O-acyl substitution was determined by a calorimetric assay described in McComb et al. (1957) Anal. Chem. 29:819-821. A low acyl mutant (LAM-1) was fermented in 100 ml salts medium in 500 ml shake flasks and gellan gum recovered. Neutral sugars and organic acid content were determined by HPLC analysis of trifluoroacetic acid hydrolysates. Results show that the mutant is deficient in addition of acetyl to gellan, while the glyceryl level is comparable to that of the control (see Exp. 1 below in Table 1). A subsequent experiment (Exp. 2 in Table 1) showed that the low level of acetyl was similar to that of a chemically deacylated purified gellan gum sample available under the tradename KELCOGEL (CP Kelco, San Diego, Calif.).

TABLE 1
LAM-1 Acyl Analysis.

| | | | | |
|---|---|---|---|---|
| | Percent | Percent | Percent | Percent |
| Strain | Rhamnose | Glucose | Glycerate | Acetate |

Exp. 1

| | | | | |
|---|---|---|---|---|
| S-60wtc | 13 | 21 | 3.4 | 2.8 |
| LAM-1 | 13 | 21 | 4.0 | 0.4 |

Exp. 2

| | | | | |
|---|---|---|---|---|
| S-60wtc | 12 | 27 | 7.9 | 2.9 |
| LAM-1 | 12 | 26 | 8.7 | 0.2 |
| KELCOGEL | 18 | 28 | 0.3 | 0.2 |

| | | |
|---|---|---|
| | Glycerate per | Acetate per |
| Strain | Repeat Unit | Repeat Unit |

Exp. 1

| | | |
|---|---|---|
| S-60wtc | 0.4 | 0.6 |
| LAM-1 | 0.5 | 0.1 |

Exp. 2

| | | |
|---|---|---|
| S-60wtc | 0.80 | 0.51 |
| LAM-1 | 0.84 | 0.03 |
| KELCOGEL | 0.02 | 0.02 |

### EXAMPLE 2

Identification and Inactivation of the Gene for Acetyl Transferase

This example demonstrates that there is a specific gene encoding the protein that catalyzes acetylation of gellan gum.

The gene for acetyl transferase was identified by complementation of the LAM-1 mutant. A gene library of Sphingomonas elodea DNA was constructed by ligating a partial PstI digest of genomic DNA into the PstI site of pLAFR3, a broad host range cosmid vector, conferring tetracycline resistance. Staskawicz et al. (1987) J. Bacteriol. 169:5789-94. This ligation mixture was transformed into E. coli strain DH5αMCR (Life Technologies Gibco BRL, Rockville, Md.). The library was then transferred into LAM-1 by triparental conjugal mating. The vector used for gene library construction was mobilizable but not self-transmissible. Transfer functions were provided by a second plasmid pRK2013 in E. coli strain JZ279. Ditta et al. (1980) Proc. Natl. Acad. Sci. USA 77:77347-7351. Strains were grown overnight in selective media: S-60 gene library in E. coli in 5 ml LB medium with tetracycline (10 μg/ml); JZ279/pRK2013 in 5 ml LB medium with kanamycin (50 μg/ml); and LAM-1 in 15 ml YEME medium. LB media contains 10 g/l tryptone, 5 g/l yeast extract and 10 g/l NaCl; YEME media contains 2.5 g/l yeast extract and 0.25 g/l malt extract. E. coli strains were concentrated two-fold and LAM-1 ten-fold. Then, 1 ml of each strain was mixed and collected on a sterile filter membrane. This membrane was transferred to a LB plate and incubated for 7 hours at 36° C. Cells were then scraped off the filter and stored in distilled water and glycerol. This culture yielded about 10⁶ cells/ml when plated on selective medium: YM medium with streptomycin (25 μg/ml, to counterselect E. coli) and tetracycline (5 μg/ml to select for plasmid containing strains). YM medium contains 3 g/l yeast extract, 3 g/l malt extract, 5 g/l peptone and 10 g/l glucose.

The LAM-1 plasmid-containing strains were then tested for acetyl composition. Each isolate was run through a three stage fermentation protocol. A colony of each test strain was inoculated into 1 ml of YM media in a 24 well Costar dish and incubated overnight at 30° C. with shaking at 250 rpm. Then 50 μl of each culture was transferred to 1 ml of salts media in a Costar dish and incubated for about 24 hours with shaking at 250 rpm at 30° C. A 0.1 ml aliquot of these cultures was used to inoculate 2.5 ml of salts media in four dram shell vials, containing ceramic balls to facilitate mixing. These were shaken at 350 rpm for about 72 hours at 36° C. The fermentation broth was hydrolyzed with 2ml of 1M trifluoroacetic acid at 90° C. for about 16 hours. A 1 ml aliquot of hydrolyzed broth was mixed with 4.5 ml of 0.137 mg/ml propionic acid (internal standard). Acyl composition was determined by high performance ion-exclusion chromatography with chemically suppressed conductivity detection, using a Dionex BioLC system. (Dionex). Salts media contains 0.229 g/l NaCl, 0.165 g/l CaCl₂.2H₂O, 2.8 g/l K₂HPO₄, 1.2 g/l KH₂PO₄, 1.9 g/l NaNO₃, 1.0 g/l NZAmine (EKC), 36.46 g/l Star Dri corn syrup, 2.5 mg/l FeSO₄.7H₂O, 24 μg/l Co₂Cl.6H₂O, and 0.101 g/l MgSO₄.7H₂O.

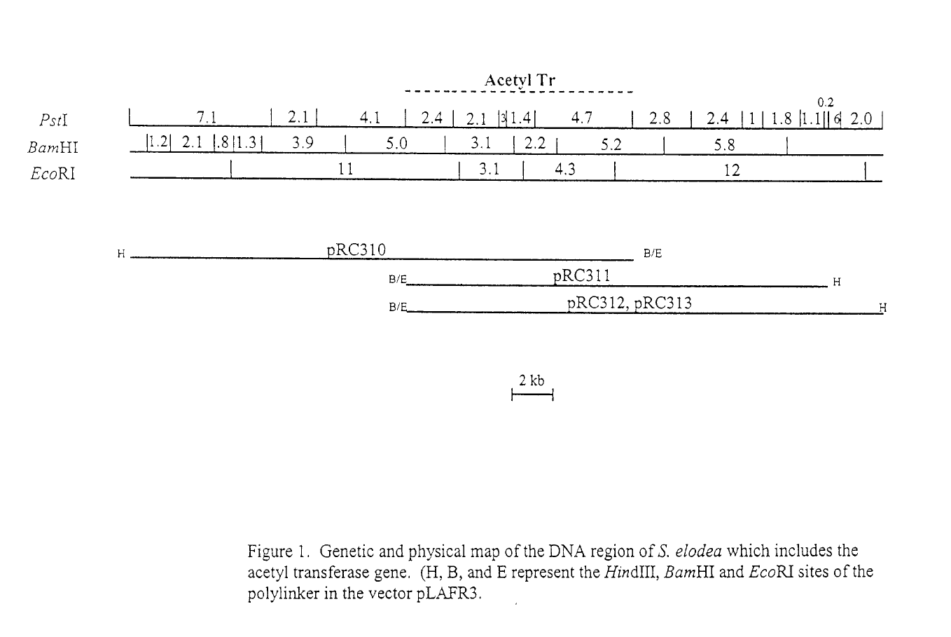

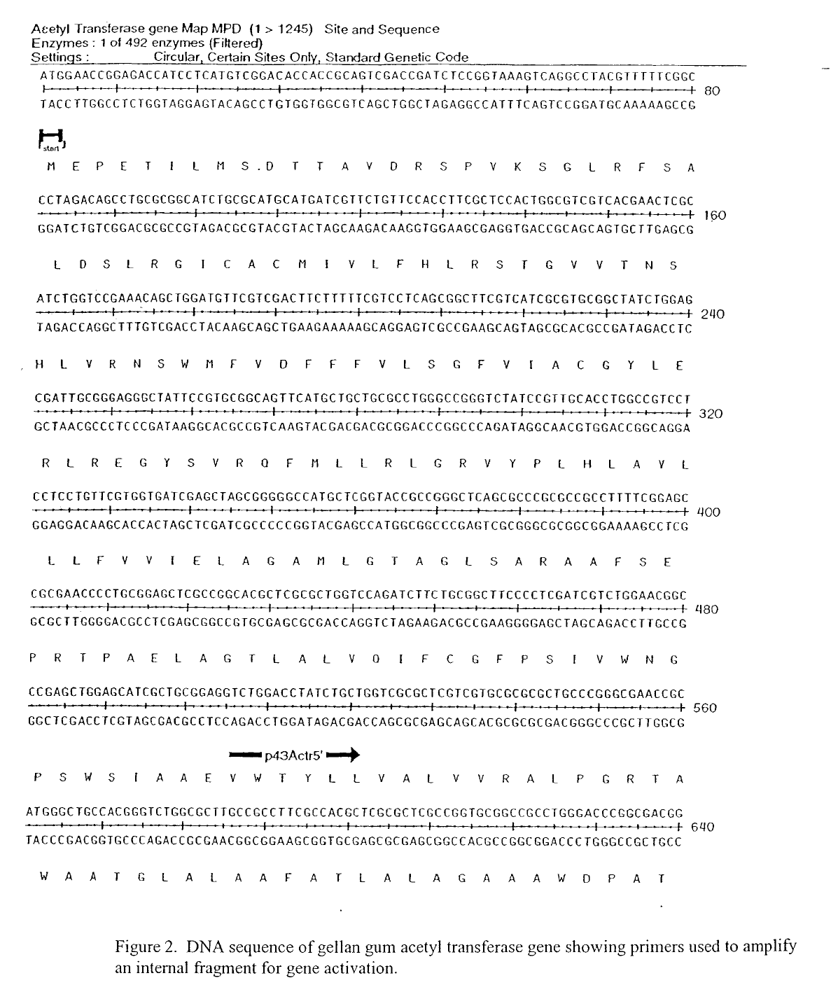

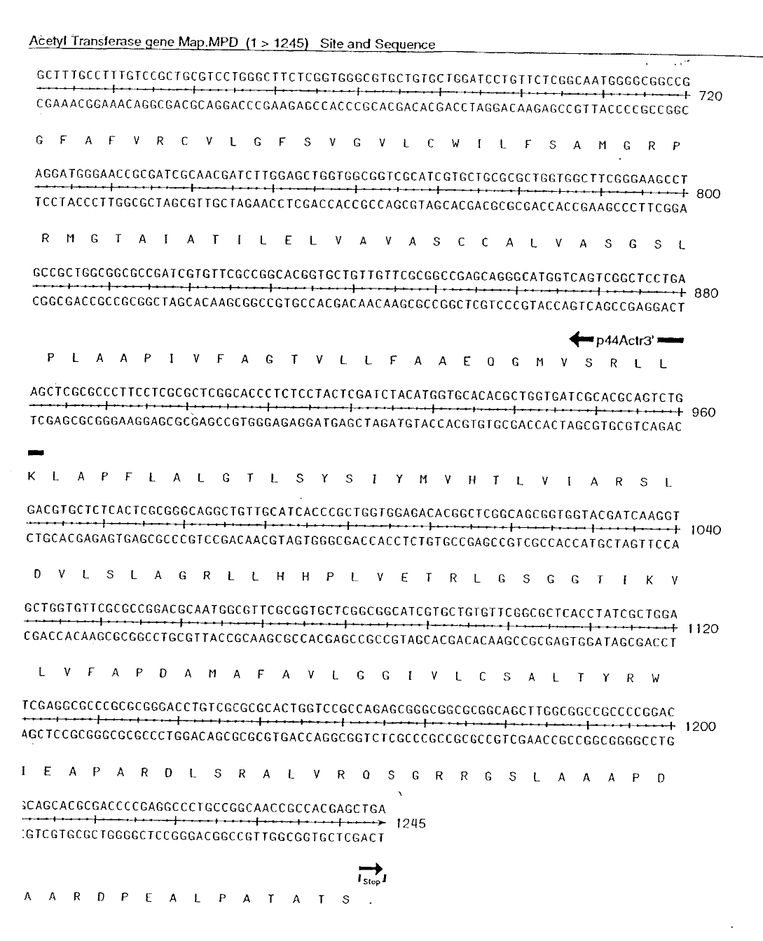

From a screen of 1398 plasmid-containing strains, four plasmids were obtained that restored acetyl substitution to gellan gum. These plasmids had 11 kb of DNA in common, as shown in FIG. 1. The sequence of this region was determined. On the 2.2 kb and 5.2 kb BamHI fragments, a gene was located which had homology to other known acetyl transferases. The gene sequence (SEQ ID NO: 1) and the protein sequence (SEQ ID NO: 2) of acetyl transferase are shown in FIG. 2.

The putative acetyl transferase gene was inactivated. Primers were designed to amplify an internal portion of the putative acetyl transferase gene. Nucleotides encoding restriction sites XbaI and SacI (underlined) were added to the ends of the PCR primers:

p43Actr5′→TTG GAG CTC TCT GGA CCT ATC TGC T
p44Actr3′→GTT TCT AGA CTT CAG GAG CCG ACT G

Primers P43Actr5′ (SEQ ID NO: 3) and P44Actr3′ (SEQ ID NO: 4), plasmid pRC311 as a template, and Ampli Taq DNA polymerase were employed in a PCR reaction to amplify the 377 base pair internal fragment of the putative acetyl transferase gene. Thirty-five PCR cycles consisting of denaturation at 96° C., annealing at 68° C. and extension at 72° C. were used to amplify the expected DNA sequence. The PCR product was digested with XbaI and SacI and ligated into similarly digested plasmid pLO2. This plasmid confers kanamycin resistance, has a site for mobilization and can replicate in E. coli but not S. elodea. Lenz et al.(1994) J. Bacteriol. 176:4385-4393.

The plasmid was transferred to S. elodea by conjugation using triparental matings. Since the plasmid cannot replicate in S. elodea, selection for kanamycin (7.5 μg/ml) resistance selects for those colonies in which the plasmid has integrated into the homologous region of the chromosome. This results in insertion of the plasmid into the putative acetyl transferase gene. Kanamycin resistant colonies were selected, purified and tested in fermentation. Analysis of the composition of gellan gum by HPLC assay of fermentation broth samples after hydrolysis with trifluoroacetic acid showed that acetyl substitution was substantially reduced, thus confirming that this is the gene that controls acetylation of the gellan polysaccharide.

TABLE 2
Gellan Broth O-Acyl Analysis.

| strain | % glycerate | % acetate |
|---|---|---|
| S-60wtc | 5.7 | 3.8 |
| S-60wtc | 5.8 | 3.9 |
| LAM-1 | 6.6 | 0.3 |
| LAM-1 | 6.1 | 0.2 |
| S-60wtc::pLO2AT-1 | 5.5 | 0.2 |
| S-60wtc::pLO2AT-1 | 5.9 | 0.4 |

LAM-1 and S-60wtc::pL02AT-1 were placed on deposit with the American Type Culture Collection under Accession Nos. PTA-4386 and PTA-4387.

Other variations and modifications of this invention will be obvious to those skilled in the art. This invention is not limited except as set forth in the claims.

What is claimed is:

1. A mutant Sphingomonas elodea capable of producing a polysaccharide polymer with substantially reduced levels of acetate.

2. The mutant Sphingomonas elodea according to claim 1 wherein said mutant Sphingomonas elodea is an acetyl transferase deficient mutant of Sphingomonas elodea.

3. A process for producing a Sphingomonas elodea polysaccharide with substantially reduced levels of acetate, said process comprising:
(a) obtaining a mutant Sphingomonas elodea which is capable of producing said polysaccharide polymer; and
(b) culturing said mutant Sphingomonas elodea under conditions effective to produce said polysaccharide polymer.

4. The process according to claim 3 wherein said mutant Sphingomonas elodea is an acetyl transferase deficient mutant of Sphingomonas elodea.

5. The process according to claim 3, further comprising the step of recovering said polysaccharide polymer.

6. A Sphingomonas elodea polysaccharide polymer with substantially reduced levels of acetate, wherein said polysaccharide polymer is produced by a process comprising the steps of:
(a) obtaining a mutant Sphingomonas elodea which is capable of producing said polysaccharide polymer; and
(b) culturing said mutant Sphingomonas elodea under conditions effective to produce said polysaccharide polymer.

7. A nucleotide sequence encoding acetyl transferase wherein said sequence comprises the DNA sequence set forth in SEQ ID NO: 1.

8. A composition comprising:
(a) a Sphingomonas elodea polysaccharide polymer with substantially reduced levels of acetate; and
(b) water.

9. The composition according to claim 8, further comprising a gelling salt.

10. The composition according to claim 8, wherein said gelling salt is selected from the group consisting of a calcium salt, a potassium salt and a sodium salt.

11. The composition according to claim 10, further comprising a sequestrant.

12. The composition according to claim 11, wherein said sequestrant is sodium citrate.

13. The composition according to claim 9, wherein said gelling salt is a calcium salt.

14. The composition according to claim 9 further comprising a fluid food product.

15. The composition according to claim 14, wherein said fluid food product is selected from the group consisting of confectionery jellies, jams, jellies, dessert gels, icings, non-dairy frozen toppings, bakery fillings and dairy products.

16. The mutant Sphingomonas elodea microorganism LAM-1 having ATCC Accession No. PTA-4386.

17. The mutant Sphingomonas elodea microorganism S-60wtc::pL02AT-1 having ATCC Accession No. PTA-4387.
